(19) United States
(12) Patent Application Publication
(10) Pub. No.: US 2009/0087909 A1
(43) Pub. Date: Apr. 2, 2009

(54) Use of Trehalose in Conifer Somatic Embryogenesis to Increase Germination Vigor

(75) Inventors: Carolyn V. Carpenter, Seattle, WA (US); Mayumi Ikeda, Tacoma, WA (US)

Correspondence Address: WEYERHAEUSER COMPANY;INTELLECTUAL PROPERTY DEPT., CH 1J27, P.O. BOX 9777, FEDERAL WAY, WA, 98063, US

(73) Assignee: Weyerhaeuser Company, Federal Way, WA (US)

(21) Appl. No.: 12/239,488

(22) Filed: Sep. 26, 2008

Related U.S. Application Data
(60) Provisional application No. 60/975,859, filed on Sep. 28, 2007.

(51) Int. Cl. C12N 5/04

(52) U.S. Cl. 435/422

(57) ABSTRACT

In one aspect, a method is provided for increasing germination vigor of conifer somatic embryos produced in vitro. The method comprises (a) culturing a plurality of immature conifer somatic embryos for a first incubation period in, or on, a first development medium that comprises less than 0.1% trehalose; and (b) culturing the plurality of immature conifer somatic embryos treated in accordance with step (a) for a second incubation period in, or on, a second development medium that comprises at least 0.1% trehalose.

### FIELD OF THE INVENTION

The present invention relates to methods for increasing germination frequency and vigor by incubating immature conifer somatic embryos in development media comprising trehalose.

### BACKGROUND OF THE INVENTION

The demand for coniferous trees, such as pines and firs, to make wood products continues to increase. One proposed solution to the problem of providing an adequate supply of coniferous trees is to identify individual coniferous trees that possess desirable characteristics, such as a rapid rate of growth, and to produce numerous, genetically identical, clones of the superior trees by somatic cloning.

Somatic cloning is the process of creating genetically identical trees from tree somatic tissue. Tree somatic tissue is tree tissue other than the male and female gametes. In one approach to somatic cloning, tree somatic tissue is cultured in an initiation medium which includes hormones, such as auxins and/or cytokinins, that initiate formation of embryogenic cells that are capable of developing into somatic embryos. The embryogenic cells are then further cultured in a maintenance medium that promotes multiplication of the embryogenic cells to form pre-cotyledonary embryos (i.e., embryos that do not possess cotyledons). The multiplied embryogenic cells are then cultured in a development medium that promotes development and maturation of cotyledonary somatic embryos which can, for example, be placed within artificial seeds, for example, as described in U.S. Pat. No. 7,131,234 and U.S. Pat. No. 5,451,241, each incorporated herein by reference, and sown in the soil where they germinate to yield conifer seedlings. The seedlings can be transplanted to a growth site for subsequent growth and eventual harvesting to yield lumber or wood-derived products. Alternatively, the cotyledonary somatic embryos can also be germinated in a germination medium, and thereafter transferred to soil for further growth.

A continuing problem with somatic cloning of conifer embryos is stimulating efficient and cost-effective formation of somatic embryos that are capable of germinating to yield plants. Preferably, conifer somatic embryos formed in vitro are physically and physiologically similar, or identical, to conifer zygotic embryos formed in vivo in conifer seeds. There is, therefore, a continuing need for methods for producing viable conifer somatic embryos from conifer embryogenic cells.

### SUMMARY OF THE INVENTION

In one aspect, a method is provided for increasing germination vigor of conifer somatic embryos produced in vitro. The method comprises (a) culturing a plurality of immature conifer somatic embryos for a first incubation period in, or on, a first development medium that comprises less than 0.1% trehalose; and (b) culturing the plurality of immature conifer somatic embryos treated in accordance with step (a) for a second incubation period in, or on, a second development medium that comprises at least 0.1% trehalose.

In another aspect, a method is provided for producing mature conifer somatic embryos. The method according to this aspect of the invention comprises (a) culturing a plurality of immature conifer somatic embryos for a first incubation period in, or on, a first development medium that comprises less than 0.1% trehalose for a first incubation period of from 6 to 8 weeks; (b) singulating a plurality of individual immature conifer somatic embryos cultured according to step (a); and (c) contacting the plurality of singulated immature conifer somatic embryos for a second incubation period in, or on, a second development medium that comprises at least 0.1% trehalose for a second incubation period sufficient in length for at least a portion of the singulated immature conifer somatic embryos to reach anatomical maturity.

The methods of the present invention are useful for preparing mature conifer somatic embryos with increased germination frequency and vigor that can be further characterized, such as by genetic or biochemical means, and/or can be germinated to produce conifer trees, if so desired. Thus, for example, the methods of the invention can be used to more efficiently produce clones of individual conifers that possess one or more desirable characteristics, such as a rapid growth rate or improved wood quality.

### BRIEF DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIG. 1 is a diagrammatic representation of early and late stage development of conifer somatic embryos; and

FIG. 2 is a schematic diagram of the metabolic pathway of trehalose.

### DETAILED DESCRIPTION

Unless specifically defined herein, all terms used herein have the same meaning as they would to one skilled in the art of the present invention.

As used herein, the term “development stage” refers to the period during somatic cloning during which histogenesis and growth of tissues and organs occurs in an immature embryo to reach a full-sized mature embryo capable of germination into a plant.

As used herein, the term “immature embryo” refers to an embryo that is not yet capable of germination into a plant, and includes embryos in early stage development (i.e., pre-cotyledonary embryos) and mid-stage development (i.e., embryos with cotyledons or hypocotyls that are not yet fully developed).

As used herein, the term “anatomical maturity” refers to an embryo that possesses developed cotyledons and hypocotyl.

As used herein, the term “cotyledonary embryo” refers to an embryo with a well-defined, elongated bipolar structure with latent meristematic centers having one or more clearly visible cotyledonary primordia at one end and a latent radicle at the opposite end.

As used herein, the term “pre-cotyledonary embryo” refers to an embryo that does not yet have cotyledons.

As used herein, the term “normal germinant” denotes the presence of all expected parts of a plant at time of evaluation. The expected parts of a plant may include a radicle, a hypocotyl, one or more cotyledon(s), and an epicotyl. In the case of gymnosperms, a normal germinant is characterized by the radicle having a length greater than 3 mm and no visibly discernable malformations compared to the appearance of embryos germinated from natural seed.

As used herein, the term “radicle” refers to the part of a plant embryo that develops into the primary root of the resulting plant.

As used herein, the term “hypocotyl” refers to the portion of a plant embryo or seedling located below the cotyledons but above the radicle.

As used herein, the term “epicotyl” refers to the portion of the seedling stem that is above the cotyledons.

As used herein, the term “embryonal suspensor mass” or “ESM” refers to a cell mass plated onto the surface of nutrient medium contained either in a semi-solid gel or as a liquid in a porous matrix capable of providing physical support, and left to grow for a period of up to three months. During the three-month incubation time, somatic embryos grow from microscopic precursor cell groups into visible early stage embryos and eventually to anatomically mature embryos. The structure of the ESM after several weeks of incubation typically consists of a proliferated mat with a few embryos sitting in direct contact with media, but most embryos forming on the top or side of the still proliferating cell mass.

As used herein, the term “trehalose” refers to α-D-glucopyranosyl-[1,1]-α-D-glucopyranoside which is a non-reducing disaccharide containing two D-glucose molecules bound in a 1,1 linkage.

As used herein, the term “trehalose anomers” refers to the three anomers of trehalose (α,β-1,1-; β,β-1,1-; and α,α-1,1-).

Unless stated otherwise, all concentration values that are expressed as percentages are weight per volume percentages.

In accordance with the methods of the invention, it has been unexpectedly discovered that culturing immature conifer somatic embryos in a first development medium that contains less than 0.1% trehalose for a first incubation period followed by culturing the immature embryos in a second development medium that contains at least 0.1% trehalose for a second incubation period, produces mature embryos that germinate at an increased frequency and/or with enhanced vigor, as compared to embryos that are incubated in development media that does not contain trehalose, as described in more detail in Examples 2-5. It has also been unexpectedly observed that the incubation of somatic conifer embryos in a second development medium containing a combination of at least 0.1% trehalose and 1% to 4% sucrose produces a synergistic effect, resulting in a dramatic increase in germination frequency of the embryos as compared to those incubated in development medium with no trehalose, or with at least 0.1% trehalose and no sucrose, as described in Examples 2-4.

In accordance with the foregoing, in one aspect, a method is provided for increasing germination vigor of conifer somatic embryos produced in vitro. The method comprises (a) culturing a plurality of immature conifer somatic embryos for a first incubation period in, or on, a first development medium that comprises less than 0.1% trehalose; and (b) culturing the plurality of immature conifer somatic embryos treated in accordance with step (a) for a second incubation period in, or on, a second development medium that comprises at least 0.1% trehalose.

The methods of the invention can be used to produce cotyledonary somatic embryos from any conifer, such as members of the genus Pinus, such as Loblolly pine (Pinus taeda) and Radiata pine. Again, by way of example, Douglas-fir embryos can be produced by the methods of the invention.

A population of mature conifer somatic embryos produced according to the methods of the invention has a greater efficiency of germinating into conifer plants than a population of conifer somatic embryos produced according to an otherwise identical control method that does not include the step of incubating the immature embryos during development in a development medium containing at least 0.1% trehalose.

In accordance with the methods of the invention, prior to incubation in a second development medium containing at least 0.1% trehalose, a first culture of immature somatic conifer embryos is incubated in a first development media containing less than 0.1% trehalose (or not containing trehalose), for a first incubation period.

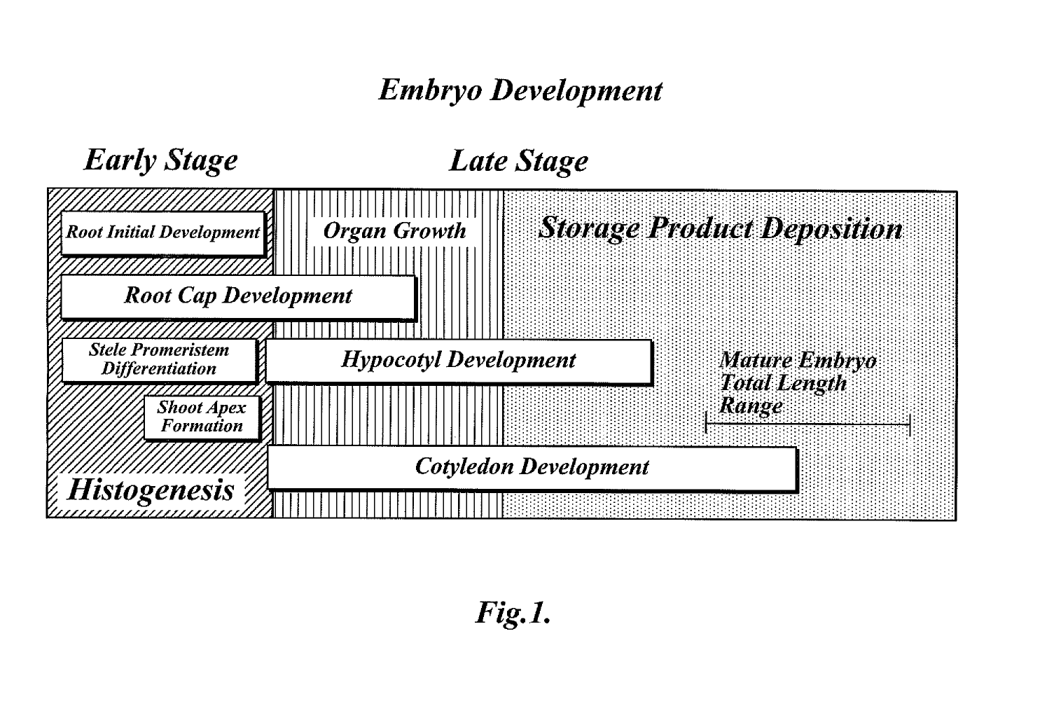

As shown in FIG. 1, the development stage of somatic conifer embryos may be divided into the early stage which involves histogenesis (i.e., the formation of different tissues from undifferentiated cells), mid-stage which involves organ growth and the initiation of hypocotyl development and cotyledon development, and the late stage which involves the completion of organ growth, the completion of hypocotyl and cotyledon development (i.e., anatomical maturity), and storage product deposition. In particular, early stage development of an immature conifer somatic embryo includes root initial development, the beginning of root cap development, stele promeristem differentiation, and shoot apex formation. Mid-stage development includes the initiation of hypocotyl development and cotyledon development, and late stage development includes completion of hypocotyl development and cotyledon development, resulting in an anatomically mature embryo.

In accordance with the methods of the invention, immature conifer somatic embryos, such as, for example, pre-cotyledonary conifer somatic embryos, can be prepared from conifer somatic cells, such as cells obtained from conifer embryos. For example, cells from conifer embryos can be induced by hormones to form embryonal suspensor cell masses (ESMs) that can be treated in accordance with the present invention to yield mature conifer somatic embryos. ESMs can be prepared, for example, from pre-cotyledonary embryos removed from seed. For example, the seeds are surface sterilized before removing the pre-cotyledonary embryos, which are then cultured on, or in, an induction medium that permits formation of ESMs which include early stage embryos in the process of multiplication by budding and cleavage. ESMs are typically cultured in a maintenance medium to form pre-cotyledonary somatic embryos. Non-limiting examples of ESM culture conditions and suitable induction and maintenance media are further described below.

In one embodiment of the method of the invention, a first culture comprising immature embryos, such as ESM comprising a plurality of pre-cotyledonary somatic embryos, are cultured in, or on, a first development medium that promotes the development of cotyledonary embryos for a first incubation period prior to incubation on a second development medium that comprises at least 0.1% trehalose.

In some embodiments, the first incubation period in the first development medium is sufficient in length for the formation of at least one of the following structures on a portion (e.g., at least one embryo, at least 10% of the embryos, at least 25%, at least 50%, more than 50%, or at least 75%) of the plurality of embryos in the first embryo culture: one or more embryos with cotyledonary primordia; one or more embryos with cotyledons; one or more embryos with 4+ cotyledons; or one or more embryos with distinct cotyledons with hypocotyl and root regions present.

The formation of one or more structures on one or more embryos (e.g., cotyledonary primordia, or cotyledons) may be determined by visual inspection or imaging analysis of the cultured embryos. Visual inspection or imaging analysis may be optionally carried out under 5-10× magnification.

The first incubation period may differ, depending on the genotype. In some embodiments, the first incubation period is from at least six weeks to at least eight weeks in length, such as from seven to eight weeks. The first incubation on the first development media may be carried out at a temperature from 10° C. to 30° C., such as from 15° C. to 25° C., or such as from 20° C. to 23° C.

At the end of the first incubation period in the first development medium, for example, when the presence of one or more cotyledonary primordia is observed on a portion of embryos, or after a time period of at least six weeks, the method comprises culturing the plurality of embryos from the first culture of embryos in a second development medium comprising at least 0.1% trehalose, for a second incubation period.

According to the methods of the invention, the plurality of immature conifer somatic embryos are cultured in, or on, a second development medium comprising at least 0.1% trehalose for a second incubation period. In some embodiments, the second incubation period is sufficient in length for at least a portion (e.g., at least one embryo, at least 10% of the embryos, at least 25%, at least 50%, more than 50%, or at least 75%) of the plurality of singulated embryos to reach anatomical maturity (i.e., possessing developed cotyledons and hypocotyl).

The second incubation period may differ, depending on the genotype. In some embodiments, the second incubation period is from at least three weeks in length, such as three to five weeks. In some embodiments, the embryos are incubated for a total length of time (including the first incubation period and the second incubation period) of at least 12 weeks on development media. The second incubation on the second development media may be carried out at a temperature from 10° C. to 30° C., such as from 15° C. to 25° C., or such as from 20° C. to 23° C.

According to the methods of the invention, immature conifer somatic embryos are cultured in a first development medium that contains less than 0.1% trehalose, and are then cultured in a second development medium that contains at least 0.1% trehalose.

Trehalose (α-D-glucopyranosyl-[1,1]-α-D-glucopyranoside) is a non-reducing disaccharide containing two D-glucose molecules bound in a 1,1 linkage, and is commonly found in lower organisms such as bacteria, fungi, and invertebrates. Trehalose has been found to act as a protectant against the deleterious effects of various stresses, such as desiccation and heat stress in yeast and bacteria (see, e.g., Muller, J. et al., Plant Science 147:37-47 (1999). However, in higher plants, trehalose is generally not accumulated, and is even considered to be toxic (see Muller, J. et al.). There are three possible anomers of trehalose (α,β-1,1-, β,β-1,1-, and α,α-1,1-), however, only the latter has been isolated from living organisms. Elbein, A. D. et al., Glycobiology 13:17R-27R; 2003. Trehalose is a nonreducing sugar, not easily hydrolyzed by acid, and the glycosidic bond is not cleaved by α-glucosidase. The molecular weight and formula are 342.31 and C₁₂H₂₂O₁₁, respectively.

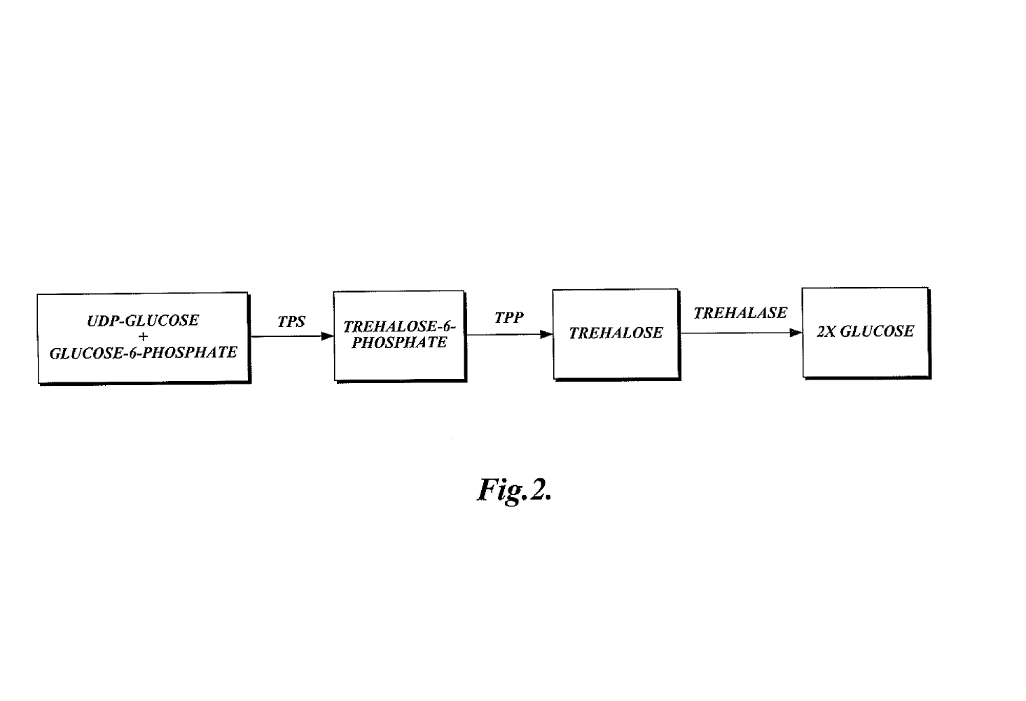

The metabolic pathway of trehalose is shown in FIG. 2. As shown in FIG. 2, Trehalose-6-phosphate synthase (TPS) catalyzes the transfer of glucose from UDP-glucose and glucose-6-phosphate to produce trehalose-6-phosphate. Trehalose-6-phosphate phosphatase (TPP) converts trehalose-6-phosphate to free trehalose. Trehalose is degraded to two molecules of glucose by trehalase.

In some embodiments of the methods of the invention, trehalose-6-phosphate is added to the second development medium in addition to trehalose, or in place of trehalose, and the plurality of immature conifer somatic embryos are incubated for the second incubation period, in order to increase germination vigor and/or germination frequency.

In accordance with the methods of the invention, the concentration of trehalose in the first development medium is less than 0.1%, such as less than 0.05%, such as less than 0.025%. In some embodiments, the first development medium does not include trehalose.

In accordance with the methods of the invention, the concentration of trehalose in the second development medium is at least 0.1%, at least 0.3%, at least 0.5%, at least 1%, at least 2%, up to 12% (such as 10%, 11% or 12%). In some embodiments, the concentration of trehalose in the second development medium is in the range of from about 0.10% to about 10.0% (such as 0.3%, 1.0%, 2.0%, 3.0%, 4.0%, 5.0%, 6.0%, 7.0%, 8.0%, or 9.0%). In some embodiments, the concentration of trehalose in the second development medium is in the range of from 0.3% to 3%.

The first and second development media typically contain nutrients that sustain the somatic embryos. Suitable development media typically do not include growth-promoting hormones, such as auxins and cytokinins.

The osmolality of the first and/or second development medium can be adjusted to a value that falls within a desired range, such as from about 250 mM/Kg to about 450 mM/Kg. Typically, an osmolality of 350 mM or higher is advantageous in the methods of the invention. An example of a suitable first development medium BM₃ is set forth in EXAMPLE 1 herein. Examples of suitable second development media are described in EXAMPLES 2-5 herein. In some embodiments of the method, the second development medium has a higher osmolality (e.g., from 350 mM/Kg to 450 mM/Kg) than the first development medium (e.g., from 300 mM/Kg to 400 mM/Kg). In some embodiments, the osmolality of the second development medium is chosen to match the osmolality of the first development medium at the end of the first incubation period.

In some embodiments, the first and/or second development medium comprises PEG at a concentration from 1% to 15%. In some embodiments, the first development medium comprises PEG at a concentration of 7% to 10% (e.g., 7%, 8%, 9%, 10%). In some embodiments, the second development medium comprises PEG at a concentration of 8% to 15% (e.g., 8%, 9%, 10%, 11%, 12%, 13%, 14%, 15%). In some embodiments, the second development medium comprises PEG at a higher concentration than the first development medium.

Maltose may be included in the first and/or second development medium as the principal or sole source of sugar for the somatic embryos. Useful maltose concentrations are within the range of from about 1% to about 4% (such as 1%, 2%, 3% or 4%).

Sucrose may be included in the first and/or second development medium. Useful sucrose concentrations are within the range of from about 1% to about 4% (such as 1%, 2%, 3% or 4%).

Glucose may be included in the first and/or second development medium. Useful glucose concentrations are within the range of from about 1% to about 4% (such as 1%, 2%, 3% or 4%).

In one embodiment of the method, the first development medium contains less than 0.1% trehalose, and includes 1% glucose and 2.5% maltose.

In one embodiment of the method, the second development medium contains trehalose at a concentration from 0.1% trehalose to 3% trehalose, and contains sucrose at a concentration from 1.0% to 4.0%, and does not contain maltose or glucose, or contains less than 0.1% maltose or 0.1% glucose. In a particular embodiment, the sugar in the second development medium consists of trehalose at a concentration from 0.1% to 3% and sucrose at a concentration from 1% to 4%.

The first and/or second development medium may contain gellan gum. Gellan gum is a gelling agent marketed, for example, under the names GELRITE and PHYTAGEL. If gellan gum is included in the development medium, it is typically present at a concentration less than about 0.5%, typically at a concentration from about 0% to about 0.4%. The first and second development media are typically a solid medium, although one or both can be a liquid medium.

The first and/or second development medium may contain an absorbent composition, such as activated charcoal, as described herein, for the induction medium.

In some embodiments, the first and/or second development medium further comprises abscisic acid. The concentration of abscisic acid in the first and/or second development medium may be between 0.5 m/gL and 500 mg/L. In some embodiments of the methods of the invention, the concentration of abscisic acid in the first and/or second development medium is between 1 mg/L and 100 mg/L. In some embodiments, the concentration of abscisic acid in the first and/or second development medium is between 5 mg/L and 25 mg/L (such as 5 mg/L to 25 mg/L, 10 mg/L to 25 mg/L, 15 mg/L to 25 mg/L).

In some embodiments, the first and/or second development medium comprises abscisic acid at a concentration from 10 mg/L (10 ppm) to 25 mg/L (25 ppm).

In some embodiments, the method further comprises the step of culturing the plurality of immature conifer somatic embryos cultured in the first development medium for a first incubation period, followed by culturing the plurality of immature conifer somatic embryos in, or on, an intermediate development medium for an intermediate incubation period before culturing the embryos in the second development medium. The intermediate incubation period in the intermediate development medium may be used to expose the immature embryos to a pulse of abscisic acid. For example, the embryos may be exposed to a high concentration of ABA in the first development medium (e.g., from 10 to 25 ppm), a reduced amount of ABA in the intermediate development medium (e.g., less than 10 ppm or no ABA), followed by exposure to the same, or greater level of ABA in the second development medium (e.g., from 5 to 25 ppm).

In accordance with this embodiment of the method, the intermediate development medium contains less than 0.1% trehalose and comprises abscisic acid at a concentration less than the first development medium. For example, the first development medium may contain abscisic acid at a concentration of from 10 ppm to 25 ppm, and the intermediate development medium may contain abscisic acid at a concentration of less than 10 ppm. In a further embodiment of the method, the second development medium may contain abscisic acid at a concentration of from 5 to 25 ppm.

In accordance with this embodiment of the methods of the invention, the combination of the first incubation period and the intermediate incubation period in total provides a time period sufficient in length for the formation of at least one of the following structures on a portion (e.g., at least one embryo, at least 10% of the embryos, at least 25%, at least 50%, more than 50%, or at least 75%) of the plurality of embryos in the intermediate embryo culture: one or more embryos with cotyledonary primordial one or more embryos with cotyledons; one or more embryos with 4+ cotyledons; or one or more embryos with distinct cotyledons with hypocotyl and root regions present.

The intermediate incubation period in the intermediate development medium may differ depending on the genotype. In some embodiments, the intermediate incubation period is from at least 3 weeks to 5 weeks, such that the combined first incubation period and intermediate incubation period is from at least six weeks to at least eight weeks in length, such as from seven to eight weeks. The intermediate incubation on the intermediate development medium may be carried out at a temperature from 10° C. to 30° C., such as from 15° C. to 25° C., or such as from 20° C. to 23° C.

In accordance with the methods of the invention, at the end of the first incubation period, or at the end of the intermediate incubation period, for example, when the presence of one or more cotyledonary primordia is observed on a portion of embryos, or after a time period of at least six weeks, the method comprises the step of incubating the embryos in the second development media. The media change may be accomplished using any suitable method, such as filtering the embryos, physically transferring the embryos from the first to the second medium, or centrifuging the embryos in the first medium, pouring off the medium and resuspending the embryos in the second medium.

In one embodiment, the method further comprises the step of singulating a plurality of embryos from the first development medium (or intermediate development medium) prior to incubating the singulated embryos on the second development medium. Any means of physically separating individual embryos from the first development culture (or intermediate development culture) of embryos may be used to singulate the embryos in accordance with this embodiment of the method of the invention. For example, physical methods of separation may be used, such as spray singulation via pressure-controlled spray of aqueous liquid, vacuuming, vibration, or picking the embryos from the first culture. Other non-limiting examples of useful singulation methods include filtering or sorting embryos based on a physical attribute such as size, shape, for example, through a sieve, or based on other physical attributes such as surface roughness, hydrophobicity, density or mass.

In some embodiments of the method, the picked embryos are laid out directly onto the surface of the second development medium, or onto a porous substrate in contact with the second development medium, which may be in solid or liquid form.

A porous substrate that is useful in the practice of various embodiments of the methods of the invention typically has a pore diameter in the range of from about 5 microns to about 1200 microns, such as from about 50 to 500 microns, such as from about 70 to about 150 microns, such as about 100 microns. The porous material is typically planar and may be any desired shape or dimension chosen for ease of manipulation and for placement in contact with the second development medium. Exemplary porous materials include materials that are sterilizable and sufficiently strong to resist tearing when the materials are lifted in order to transfer singulated embryos to subsequent stages of the somatic embryo production process, such as stratification. Examples of useful porous materials include, but are not limited to, membranes, nylon fiber, woven mesh (e.g., nylon, stainless steel or plastic), and polymeric fibers.

In some embodiments, the singulated embryos are transferred to a second development medium, or a porous substrate in contact with a second development medium, in such a manner that the singulated embryos are not in physical contact with one another.

As described above, according to the methods of the invention, after singulation, the singulated immature embryos are contacted with the second development medium for a second incubation period. In some embodiments, the second incubation period is sufficient in length for at least a portion (e.g., at least one embryo, at least 10% of the embryos, at least 25%, at least 50%, more than 50%, or at least 75%) of the plurality of singulated embryos to reach anatomical maturity (i.e., possessing developed cotyledons and hypocotyl).

In some embodiments of the method, after incubation in the second development medium, the embryos are then cultured in, or on, a stratification medium for a period of about one week to about six weeks, at a temperature of from about 1° C. to about 10° C. Typically, the stratification medium is similar or identical to the first development medium, but does not contain abscisic acid and has a lower concentration of gellan gum, typically less than about 0.5%. The stratification medium may contain sucrose as the principal or sole source of metabolizable sugar. An exemplary stratification medium is set forth as BM₄ in EXAMPLE 1.

In some embodiments, the present invention provides methods for producing mature conifer somatic embryos, comprising the steps of (a) culturing a plurality of immature conifer somatic embryos for a first incubation period in, or on, a first development medium that comprises less than 0.1% trehalose for a first incubation period of from 6 to 8 weeks; (b) singulating a plurality of individual immature conifer somatic embryos cultured according to step (a); and (c) contacting the plurality of singulated immature conifer somatic embryos for a second incubation period in, or on, a second development medium that comprises at least 0.1% trehalose for a second incubation period sufficient in length for at least a portion of the singulated immature conifer somatic embryos to reach anatomical maturity.

In some embodiments, the method further comprises culturing conifer somatic cells in, or on, an induction medium to yield embryogenic cells, followed by culturing the embryogenic cells in, or on, a maintenance medium prior to the step of incubating the immature conifer somatic embryos in, or on, the first development medium comprising less than 0.1% trehalose.

Embryogenic cells are cells that are capable of producing one or more cotyledonary conifer somatic embryos and include, for example, conifer embryonal suspensor masses. The induction medium typically includes inorganic salts and organic nutrient materials. The osmolality of the induction medium is typically about 160 mg/kg or even lower, but it may be as high as 170 mM/kg. The induction medium typically includes growth hormones. Examples of hormones that can be included in the induction medium are auxins (e.g., 2,4-dichlorophenoxyacetic acid (2,4-D)) and cytokinins (e.g., 6-benzylaminopurine (BAP)). Auxins can be utilized, for example, at a concentration of from 1 mg/L to 200 mg/L. Cytokinins can be utilized, for example, at a concentration of from 0.1 mg/L to 10 mg/L.

The induction medium may contain an absorbent composition, especially when very high levels of growth hormones are used. The absorbent composition can be any composition that is not toxic to the embryogenic cells at the concentrations utilized in the practice of the present methods and that is capable of absorbing growth-promoting hormones and toxic compounds produced by the plant cells during embryo development that are present in the medium. Non-limiting examples of useful absorbent compositions include activated charcoal, soluble poly(vinyl pyrrolidone), insoluble poly(vinyl pyrrolidone), activated alumina, and silica gel. The absorbent composition may be present in an amount, for example, of from about 0.1 g/L to about 5 g/L. An example of an induction medium useful in the practice of the present invention is medium BM₁ set forth in EXAMPLE 1 herein. The induction medium is typically solid, and may be solidified by inclusion of a gelling agent.

Conifer somatic cells are typically cultured in, or on, an induction medium for a period of from three weeks to ten weeks, such as from six weeks to eight weeks, at a temperature of from 10° C. to 30° C., such as from 15° C. to 25° C., or such as from 20° C. to 23° C.

In accordance with the methods of this aspect of the invention, the maintenance medium may be a solid medium, or it may be a liquid medium which can be agitated to promote growth and multiplication of the embryogenic tissue. The osmolality of the maintenance medium is typically higher than the osmolality of the induction medium, typically in the range of 180-400 mM/kg. The maintenance medium may contain nutrients that sustain the embryogenic tissue, and may include hormones, such as one or more auxins and/or cytokinins, that promote cell division and growth of the embryogenic tissue. Typically, the concentrations of hormones in the maintenance medium is lower than their concentration in the induction medium.

It is generally desirable, though not essential, to include maltose as the sole, or principal, metabolizable sugar source in the maintenance medium. Examples of useful maltose concentrations are within the range of from about 1% to about 2.5%. An example of a suitable maintenance medium is medium BM₂ set forth in EXAMPLE 1 herein. Conifer embryogenic cells are typically transferred to fresh maintenance medium once per week.

As described above, immature conifer somatic cells formed from conifer embryogenic cells are cultured in, or on, a first development medium containing less than 0.1% trehalose for a first incubation period, singulated, and then cultured on a second development medium comprising at least 0.1% trehalose for a second incubation period. Useful development media and incubation time periods are described supra.

After being cultured in the second development medium with at least 0.1% trehalose, the cotyledonary somatic embryos can optionally be transferred to a stratification medium for a further period of culture as described supra.

The conifer cotyledonary somatic embryos produced using the methods of the invention can optionally be germinated to form conifer plants which can be grown into coniferous trees, if desired. The cotyledonary embryos may also be disposed within artificial seeds for subsequent germination. The conifer cotyledonary somatic embryos can be germinated, for example, on a solid germination medium, such as the germination medium described in EXAMPLE 1 herein. The germinated plants can then be transferred to soil for further growth. For example, the germinated plants can be planted in soil in a greenhouse and allowed to grow before being transplanted to an outdoor site. Typically, the conifer cotyledonary somatic embryos are illuminated to stimulate germination.

The methods of the invention may be used to produce a population of mature conifer somatic embryos with a capacity to germinate at a higher frequency (i.e., produce a higher yield of germinants) than a population of conifer somatic embryos produced according to an otherwise identical method that does not include the step of singulating immature embryos during development. Some embodiments of the methods of the invention yield mature conifer somatic embryos that have a germination efficiency that is at least 20% higher than the germination efficiency of mature conifer somatic embryos produced according to an otherwise identical method that does not include the step of incubating immature conifer somatic embryos in the development medium comprising at least 0.1% trehalose, as further described in EXAMPLES 2-5.

The following examples merely illustrate the best mode now contemplated for practicing the invention, but should not be construed to limit the invention.

### EXAMPLE 1

This Example describes a representative method for producing somatic pine embryos from Loblolly Pine using post-development singulation.

Methods:

Female gametophytes containing zygotic embryos are removed from seeds four to five weeks after fertilization. The seed coats are removed, but the embryos are not further dissected out of the surrounding gametophyte other than to excise the nucellar end. The cones are stored at 4° C. until used. Immediately before removal of the immature embryos, the seeds are sterilized utilizing an initial washing and detergent treatment followed by a ten-minute sterilization in 15% H₂O₂. The explants are thoroughly washed with sterile distilled water after each treatment.

Tables 1 and 2 set forth exemplary compositions of media useful for producing pine somatic embryos.

TABLE 1
Pinus Taeda Basal Medium (BM)

| Constituent | Concentration (mg/L) |
|---|---|
| NH₄NO₃ | 150.0 |
| KNO₃ | 909.9 |
| KH₂PO₄ | 136.1 |
| Ca(NO₃)₂•4H₂O | 236.2 |
| CaCl₂•4H₂O | 50.0 |
| MgSO₄•7H₂O | 246.5 |
| Mg(NO₃)₂•6H₂O | 256.5 |
| MgCl₂•6H₂O | 50.0 |
| KI | 4.15 |
| H₃BO₃ | 15.5 |
| MnSO₄•H₂O | 10.5 |
| ZnSO₄•7H₂O | 14.4 |
| NaMoO₄•2H₂O | 0.125 |
| CuSO₄•5H₂O | 0.125 |
| CoCl₂•6H₂O | 0.125 |
| FeSO₄•7H₂O | 27.86 |
| Na₂EDTA | 37.36 |
| Maltose | 30,000 |
| myo-Inositol | 200 |
| Casamino acids | 500 |
| L-Glutamine | 1000 |
| Thiamine-HCl | 1.00 |
| Pyridoxine-HCl | 0.50 |
| Nicotinic acid | 0.50 |
| Glycine | 2.00 |
| Gelrite⁺ | 1600 |
| pH adjusted to 5.7 | |

⁺Used if a solid medium is desired.

TABLE 2
Composition of Media for Different Stage Treatments

| | |
|---|---|
| BM₁-Induction Medium | BM + 2,4-D (15 μM) + Kinetin (2 μM) + BAP (2 μM). |
| BM₂-Maintenance Medium | BM + 2,4-D (5 μM) + Kinetin (0.5 μM) + BAP (0.5 μM). |
| | GELRITE (1600 mg/L) is added when a solid medium is |
| | desired. |
| Dilution Medium | BM + 10 ppm abscisic acid + 100-1000 mg/mL additional |
| | myo-inositol, +2.5% Maltose. The following amino acid |
| | mixture is added: L-proline (100 mg/L), L-asparagine |
| | (100 mg/L), L-arginine (50 mg/L), L-alanine (20 mg/L), and |
| | L-serine (20 mg/L). Preferably no maintenance hormones |
| | are present. |
| BM₃-Development Medium | BM + 25 mg/L abscisic acid + 12% PEG-8000 + 800 mg/L |
| | additional myo-inositol + 0.1% activated charcoal + 1% |
| | glucose, +2.5% Maltose. The following amino acid mixture |
| | is added: L-proline (100 mg/L), L-asparagine (100 mg/L), |
| | L-arginine (50 mg/L), L-alanine (20 mg/L), and L-serine |
| | (20 mg/L). GELRITE (2500 mg/L) is added when a solid |
| | medium is desired. |
| BM₄-Stratification Medium | BM₃ modified by omitting abscisic acid, and PEG-8000. |
| | GELRITE (2500 mg/L) is added when a solid medium is |
| | desired. |
| BM₅-Germination Medium | BM modified by replacing maltose with 2% sucrose. |
| | Myo-inositol is reduced to 100.0 mg/L, glutamine and |
| | casamino acids are reduced to 0.0 mg/L. FeSO₄•7H₂O is |
| | reduced to 13.9 mg/L and Na₂EDTA reduced to 18.6 mg/L. |
| | Agar at 0.8% and activated charcoal at 0.25% are added. |

Induction: Sterile gametophytes with intact embryos are placed on a solid BM₁ culture medium and held in an environment at 22°-25° C. with a 24-hour dark photoperiod for a time of 3-5 weeks. The length of time depends on the particular genotype being cultured. At the end of this time, a white mucilaginous mass forms in association with the original explants. Microscopic examination typically reveals numerous early stage embryos associated with the mass. These are generally characterized as having a long thin-walled suspensor associated with a small head with dense cytoplasm and large nuclei.

Osmolality of the induction medium may in some instances be as high as 150 mM/kg. Typically it is about 120 mM/kg or even lower (such as 110 mM/kg).

Maintenance and Multiplication of Immature (Pre-Cotyledonary) Embryos: Early stage embryos removed from the masses generated in the induction stage are placed on a gelled maintenance medium BM₂. This differs from the induction medium in that the growth hormones (auxins and cytokinins) are reduced by at least a full order of magnitude. Osmolality of this medium is at 130 mM/kg or higher (typically within the range of about 120-150 mM/kg for Pinus taeda). The temperature and photoperiod are again 22°-25° C. with 24 hours in the dark. Embryos are cultured 12-14 days on the BM₂ solid maintenance medium before transferring to a liquid medium for further subculturing. This liquid medium has the same composition as BM₂, but lacks the gellant. The immature embryos at the end of the solid maintenance stage are typically similar in appearance to those from the induction stage. After 5 to 6 weekly subcultures in the liquid maintenance medium, advanced early stage embryos have formed. These are characterized by smooth embryonal heads, estimated to typically have over 100 individual cells, with multiple suspensors.

Embryo Development:

Early stage immature embryos are transferred to a solid development medium. The development medium either lacks growth hormones entirely, or has them present only at very low levels. Abscisic acid (ABA) may be included to facilitate further development. The further inclusion of an absorbent material in this medium is advantageous. The absorbent material may be chosen from a number of chemical materials having high surface area and/or controlled pore size, such as activated charcoal, soluble and insoluble forms of poly(vinyl pyrrolidone), activated alumina, and silica gel. The absorbent composition is normally present at a concentration of about 0.1-5 g/L, more commonly at about 0.25-2.5 g/L. Gellan gum may be included at a concentration of about 0.25%.

The osmotic potential of the development medium may be raised substantially over that of the maintenance medium. It has been found advantageous to have an osmolality as high as 350 mM/kg or even higher. Development is preferably carried out in complete darkness at a temperature of 22°-25° C. until cotyledonary embryos have developed (e.g., reached anatomical maturity). Development time is typically several weeks, such as 7 to 12 weeks.

Stratification: After 7 to 12 weeks on development medium, cotyledonary embryos are singulated and transferred to stratification medium BM₄. The singulation step includes any means of physically separating individual embryos from the first culture of embryos. The stratification medium is similar to development medium, but lacks abscisic acid, PEG-8000, and gellan gum. Embryos are cultivated on stratification medium at between about 1° C. and about 10° C. in the dark for between 3 to 6 weeks.

Drying: The mature embryos are lifted from the growth substrate and placed in a closed container over H₂O at a relative humidity of 97%, for a period of from one to three weeks.

Germination: The conditioned mature embryos are rehydrated by placing them on a pad saturated with liquid germination medium. The embryos are then placed individually on solid BM₅ medium for germination. This is a basal medium lacking growth hormones which is modified by reducing sucrose, myo-inositol and organic nitrogen. The embryos are incubated on BM₅ germination medium for sufficient time, typically about 6 weeks, under environmental conditions of 23°-25° C., and a 16-hour light, 8-hour dark photoperiod, until the resulting plantlets have well developed radicle and hypocotyl, and green cotyledonary structure and epicotyl.

Because of the reduced carbohydrate concentration, the osmotic potential of the germination medium may be further reduced below that of the development medium. It is normally below about 150 mM/kg (such as about 100 mM/kg).

### EXAMPLE 2

This Example demonstrates the dramatic improvement in germination frequency observed when immature Loblolly pine embryos of Genotype A were incubated in development medium containing trehalose.

Methods:

Somatic Loblolly pine embryos of genotype A were tested in this experiment. After incubation in maintenance medium using the methods described in Example 1, advanced early stage somatic embryos of Loblolly pine, genotype A were initially plated in liquid development medium BM₃ (BM+25 mg/L ABA, 1% glucose, 2.5% maltose). Three flasks of embryos were plated (approximately 80 plates per flask), for a total of 240+ plates per genotype, per plating. At 4 and 9 weeks, the initial development medium was replaced for each treatment as described below in TABLE 3, thereby enabling the addition or removal of specific components of the development medium.

As shown in TABLE 3, at 4 weeks, the initial development medium in the experimental groups was replaced with a modified first developmental medium (BM₃ with no ABA, 1% glucose, 2.5% maltose). At 9 weeks, the embryos were singulated as indicated in TABLE 3, and the singulated embryos were transferred to a modified second developmental medium (BM₃ with 10 ppm ABA) in combination with various additions of trehalose and sucrose, shown in TABLE 3. Additionally, several treatments also reintroduced ABA at 9 weeks, creating the effect of a second pulse of ABA (see TABLE 3). The immature embryos were incubated in the second developmental medium for an additional 3 weeks. Embryos were singulated and placed in stratification medium BM₄ and cultivated for 4 weeks before drying in closed containers at approximately 97% humidity (see EXAMPLE 1).

TABLE 3
Experimental Design/Media Replacements

| Treatment | | | |
|---|---|---|---|
| (all start in initial | | | |
| development | | | |
| media: either | | | |
| semi-solid BM₃ | | | |
| or liquid BM₃ | | | |
| (+25 ppm ABA, | | | |
| no trehalose, 1% | 4 week first | 9 week second | ABA Content (ppm) |
| glucose, 2.5% | development media | development media | and trehalose Content |
| maltose) | replacement | replacement | During Experiment |
| 1 | no change - embryos | no change - embryos | ABA: 25, NC, NC |
| | stay on BM₃ (+25 ppm | stay on BM₃ | Trehalose: 0, 0, 0 |
| | ABA) | | |
| 2 | fresh BM₃ (+25 ppm | fresh BM₃ (+25 ppm | ABA: 25, 25, 25 |
| | ABA) | ABA) | Trehalose: 0, 0, 0 |
| 3 | liquid modified BM₃ | liquid modified BM₃ (+10 ppm | ABA: 25, 0, 10 |
| | (No ABA) | ABA) | Trehalose: 0, 0, 0 |
| 4 | liquid BM₃, no | no change, stays on | ABA: 25, NC, NC, |
| | change, but open lid | liquid BM₃, but open | (open lid) |
| | briefly | lid briefly | Trehalose: 0, 0, 0 |
| 5 | liquid modified BM₃ | liquid modified BM₃ | ABA: 25, 0, 10 |
| | (No ABA) | (+10 ppm ABA, | Trehalose: 0, 0, 0.3% |
| | | +0.3% trehalose,) | |
| 6 | liquid modified BM₃ | liquid modified BM₃ | ABA: 25, 0, 10 |
| | (No ABA) | (+10 ppm ABA, +1% | Trehalose: 0, 0, 1% |
| | | trehalose) | |
| 7 | liquid modified BM₃ | liquid modified BM₃ | ABA: 25, 0, 10 |
| | (No ABA) | (+10 ppm ABA, No | Trehalose: 0, 0, 0 |
| | | maltose, +3% sucrose) | Maltose: 2.5%, 2.5%, 0 |
| | | | Sucrose: 0%, 0%, 3% |
| 8 | liquid modified BM₃ | liquid modified BM₃ | ABA: 25, 0, 10 |
| | (No ABA) | (+10 ppm ABA, +0.3% | Trehalose: 0, 0, 0.3% |
| | | trehalose, No maltose, | Maltose: 2.5%, 2.5%, 0 |
| | | +3% sucrose) | Sucrose: 0%, 0%, 3% |

After the 9-week second development media replacement, the embryos were incubated for an additional 3 weeks, resulting in a total development incubation period of 12 weeks. After development, the embryos were transferred to stratification medium BM₄, and germinated on medium BM₅, as described in EXAMPLE 1. After 6 weeks incubation on germination medium, the embryos were assessed for germination rate, root length, hypocotyls length, and epicotyl length. An embryo was considered a normal germinant if it was in Class 1, which includes the following features: the presence of a 1 mm root (no nubbins), the presence of approximately 5 epicotyl leaves approximately 5 mm long, no large scale hypocotyl ruptures, and the hypocotyl not bent greater than 90 degrees.

Germination rate was analyzed with a generalized linear fixed effects model (SAS Proc Genmod). Mean comparisons were made using Fisher's least significant difference (LSD) procedures, followed by pair wise chi-squared tests. A linear mixed effects model (SAS Proc Mixed) using germination box means was applied for root length, hypocotyl length, and epicotyl length. Means comparisons were made using Tukey's procedure for multiple comparisons with α=0.10.

Results: A late maturation treatment of trehalose and sucrose was found to significantly increase the germination rate for embryos of Loblolly pine, genotype A. Results of subsequent LSD comparisons with respect to germination rates for the various treatment conditions are shown in TABLE 4. A significant variation of germination rate of genotype A embryos was detected among the treatment groups (p=0.0012). Treatments with the same symbols in the LSD column (i.e., a,b,c) were not significantly different. Specifically, a treatment of 0.3% trehalose, 3% sucrose, 1% glucose, 10 ppm ABA (treatment 8) during late maturation resulted in a significant increase in germination over controls and all other treatments. Treatments consisting of 0.3% trehalose or 1% trehalose without sucrose (treatments 5 and 6, respectively) did not have significant effects on germination. Additionally, it is noted that treatment with 3% sucrose without trehalose had no significant effect on germination, indicating an unexpected synergistic effect of the combination of sucrose and trehalose for the stimulation of germination in genotype A embryos.

TABLE 4
Germination Rates for Genotype A
Germination Rate (P-value = 0.0012)

| | LS | |
|---|---|---|
| Treatment | mean | LSD (α = 0.10) |
| 8 | 0.55 | a |
| 3 | 0.46 | b |
| 7 | 0.44 | bc |
| 5 | 0.43 | bc |
| 1 | 0.43 | bc |
| 6 | 0.42 | bc |
| 2 | 0.39 | c |
| 4 | 0.37 | c |

The results of the various treatments on measurements of germinants from genotype A are shown in TABLE 5 (hypocotyl length), TABLE 6 (root length) and TABLE 7 (epicotyl length). For root length, a low dose of trehalose without sucrose (treatment 5) stimulated significantly more root growth than other treatments utilizing trehalose or sucrose, as shown in TABLE 6. Similar results were obtained by replacing the original medium (treatment 2) or creating two pulses of ABA (treatment 3).

In contrast to the significant effects observed for germination frequency described above, the effect of the combination of trehalose and sucrose during late maturation was not as apparent for aspects organ length of genotype A embryos. For instance, the combination of trehalose and sucrose (treatment 8) did not significantly outperform the other treatments for increasing hypocotyl length, (as shown in TABLE 5), root length (as shown in TABLE 6), or epicotyl length (as shown in TABLE 7). For increasing hypocotyl length, the best treatment was merely to open the lid to create more air circulation over the developing embryos (treatment 4). Finally, there were no significant differences among the treatment groups for effecting hypocotyl length (TABLE 7).

TABLE 5
Hypocotyl Length for Genotype A
Hypocotyl Length (P-value = 0.0003)

| | LS | |
|---|---|---|
| Treatment | mean | LSD (α = 0.10) |
| 4 | 11.77 | a |
| 2 | 11.50 | ab |
| 3 | 11.28 | abc |
| 8 | 11.20 | bc |
| 5 | 10.93 | c |
| 7 | 10.86 | c |
| 1 | 10.78 | cd |
| 6 | 10.33 | d |

TABLE 6
Root Length for Genotype A
Root Length (P-value = 0.0983)

| | LS | |
|---|---|---|
| Treatment | mean | LSD (α = 0.10) |
| 5 | 27.36 | a |
| 2 | 26.15 | ab |
| 3 | 25.42 | ab |
| 8 | 24.67 | b |
| 7 | 24.49 | bc |
| 4 | 24.20 | bc |
| 1 | 23.08 | c |
| 6 | 22.96 | c |

TABLE 7
Epicotyl Length for Genotype A
Epicotyl Length (P-value = 0.3367)

| | LS | |
|---|---|---|
| Treatment | mean | LSD (α = 0.10) |
| 5 | 11.02 | NA |
| 3 | 10.84 | NA |
| 2 | 10.71 | NA |
| 4 | 10.70 | NA |
| 8 | 10.69 | NA |
| 1 | 10.68 | NA |
| 6 | 10.58 | NA |
| 7 | 9.94 | NA |

Therefore, these results indicate that the addition of trehalose during late maturation of immature somatic Loblolly pine embryos significantly enhances development and germination frequency. It was unexpectedly observed that the combination of trehalose and sucrose significantly increases the percent of embryos that germinate when applied with two ABA pulses during late maturation. For root length, a low dose of trehalose without sucrose (treatment 5) stimulated significantly more root growth than other treatments utilizing trehalose or sucrose.

### EXAMPLE 3

This Example demonstrates the dramatic improvement in germination frequency observed when immature Loblolly pine embryos of Genotype B were incubated in the development medium containing trehalose.

Methods: Somatic Loblolly pine embryos of genotype B were plated according to the general protocol described in Example 2, except that an additional, separate trial tested all treatments on embryos initially plated on semi-solid development medium BM₃. The corresponding replacement development media in that trial were also semi-solid rather than liquid. The treatments, time periods, and statistical analysis were performed as described in EXAMPLE 2.

Results: The germination rates for genotype B embryos in liquid or semi-solid development media under the different treatment conditions is provided in TABLE 8. As shown in TABLE 8, the late maturation treatment of trehalose, either alone or in combination with sucrose, was found to significantly increase the germination frequency of Loblolly pine embryos, genotype B, as compared to the basic control group where no replacement development medium was provided. A significant variation of germination rate was detected among the treatment groups, in both liquid and semi-solid development medium (p=0.0003, p<0.0001, respectively). Results of subsequent LSD comparisons are shown in TABLE 8. Treatments with the same symbols in the LSD column (i.e., a,b,c) are not significantly different. In both media trials, the best treatment for enhancing germination of genotype B embryos was to supply fresh development medium without additional additives (treatment 2). However, this effect was not observed in genotype A (see EXAMPLE 2). In the liquid development medium, however, treatment of 0.3% trehalose without sucrose (treatment 5) performed just as well. Treatment 5 resulted in a germination rate that was statistically indistinguishable from supplying fresh plating medium. Adding 0.3% trehalose increased the germination rate over the control, simulating the two pulses of ABA (treatment 3); however, the difference observed was not statistically significant.

For the semi-solid plating medium, it was observed that the addition of 1% trehalose without sucrose, and 0.3% trehalose plus 3% sucrose (treatments 6 and 8, respectively), produced results that were statistically indistinguishable from the most effective treatment (treatment 2). All treatments involving addition of trehalose significantly enhanced germination rates above the control where no new medium was provided (treatment 1). Moreover, treatments of 0.3% trehalose plus 3% sucrose (treatment 8) and 1% trehalose without sucrose (treatment 6) each increased the germination rate over the control, simulating the two pulses of ABA (treatment 3), although the differences were not significant.

TABLE 8
Germination Rates for Genotype B

| Germination Rate | | | Germination Rate | | |
|---|---|---|---|---|---|
| (P-value = 0.0003) | | | (P-value <0.0001) | | |
| liquid development media | | | semi-solid development media | | |
| | LS | | | LS | LSD |
| Treatment | mean | LSD (α = 0.10) | Treatment | mean | (α = 0.10) |
| 2 | 0.57 | a | 2 | 0.52 | a |
| 5 | 0.51 | ab | 8 | 0.47 | ab |
| 3 | 0.44 | bc | 6 | 0.47 | ab |
| 8 | 0.43 | bc | 3 | 0.44 | b |
| 4 | 0.43 | bc | 5 | 0.40 | b |
| 7 | 0.40 | bc | 7 | 0.38 | bc |
| 1 | 0.37 | c | 4 | 0.33 | cd |
| 6 | NA | NA | 1 | 0.32 | d |

The results of the various treatments on measurements of germinants from genotype B are show below in TABLE 9 (hypocotyl length), TABLE 10 (root length), and TABLE 11 (epicotyl length). As shown in TABLES 9-11, trehalose treatments were observed to have some significant positive effects on organ lengths in the germinants. For example, in a semi-solid plating medium, a 0.3% trehalose treatment (treatment 5) significantly increased hypocotyl length over the basic control, where no medium was replaced (Table 9). Also, in a semi-solid plating medium, all treatments with trehalose, whether or not in conjunction with sucrose, significantly increased epicotyl length over the basic control treatment (TABLE 11).

TABLE 9
Hypocotyl length of Genotype B

| Hypocotyl Length | | | Hypocotyl Length | | |
|---|---|---|---|---|---|
| (P-value = 0.1129) | | | (P-value = 0.0003) | | |
| liquid development media | | | semi-solid development media | | |
| | LS | | | | LSD |
| Treatment | mean | LSD (α = 0.10) | treatment | LS mean | (α = 0.10) |
| 2 | 6.85 | NA | 2 | 7.46 | a |
| 7 | 6.80 | NA | 3 | 7.39 | a |
| 3 | 6.79 | NA | 5 | 7.33 | a |
| 5 | 6.72 | NA | 7 | 7.02 | ab |
| 4 | 6.71 | NA | 8 | 6.83 | bc |
| 8 | 6.55 | NA | 6 | 6.69 | bc |
| 1 | 6.19 | NA | 4 | 6.59 | bc |
| 6 | NA | NA | 1 | 6.43 | c |

TABLE 10
Root length of Genotype B

| Root Length | | | Root Length | | |
|---|---|---|---|---|---|
| (P-value = 0.0224) | | | (P-value = 0.0421) | | |
| liquid development media | | | semi-solid development media | | |
| | LS | | | | LSD |
| Treatment | mean | LSD (α = 0.10) | treatment | LS mean | (α = 0.10) |
| 2 | 12.78 | a | 3 | 13.63 | a |
| 5 | 11.67 | ab | 4 | 13.37 | a |
| 4 | 10.92 | bc | 2 | 13.26 | ab |
| 8 | 10.48 | bc | 5 | 12.79 | abc |
| 1 | 10.19 | bc | 6 | 12.09 | bc |
| 3 | 10.02 | bc | 1 | 11.90 | c |
| 7 | 9.47 | c | 8 | 11.31 | cd |
| 6 | NA | NA | 7 | 11.23 | d |

TABLE 11
Epicotyl Length of Genotype B

| Epicotyl Length | | | Epicotyl Length | | |
|---|---|---|---|---|---|
| (P-value = 0.0001) | | | (P-value = 0.031) | | |
| liquid development media | | | semi-solid development media | | |
| | LS | | | | LSD |
| Treatment | mean | LSD (α = 0.10) | treatment | LS mean | (α = 0.10) |
| 4 | 11.30 | a | 3 | 12.21 | a |
| 2 | 11.24 | a | 2 | 11.69 | ab |
| 5 | 10.20 | b | 6 | 11.51 | ab |
| 1 | 10.09 | b | 7 | 11.32 | b |
| 8 | 9.80 | bc | 5 | 11.28 | b |
| 7 | 9.47 | bc | 8 | 11.25 | b |
| 3 | 9.06 | c | 4 | 11.12 | bc |
| 6 | NA | NA | 1 | 10.33 | c |

In summary, consistent with the results described in EXAMPLE 2 for genotype A, these data show that the addition of trehalose to late development media stimulates an increased germination rate in genotype B embryos.

### EXAMPLE 4

This Example demonstrates that the addition of trehalose to late development media significantly increases the germination rate of Loblolly pine embryos in comparison to control embryos incubated in late development medium without trehalose.

Methods: Somatic embryos of Loblolly pine, genotypes C and D, were cultured as described in EXAMPLE 1 up to incubation in the maintenance media. After incubation in maintenance media using the methods described in EXAMPLE 1, advanced early stage somatic embryos were plated on semi-solid development medium BM₃ (with 25 ppm ABA, 1% glucose and 2.5% maltose) at 25 embryos per plate, 15 plates per genotype. The subsequent experimental design is presented in TABLE 12. Specifically, at five weeks after plating, the media for 10 treatment plates per genotype were replaced with modified development media BM₃ containing 5 ppm ABA instead of 25 ppm ABA. The media for the remaining five plates per genotype were replaced with fresh development medium BM₃ for controls. At 8 weeks after initial plating, the embryos were singulated and exposed again to fresh media. For treatment 1, the media for five plates per genotype that had been incubating in the modified development medium BM₃ (containing 5 ppm ABA) were replaced with new modified development media BM₃ lacking maltose and containing 0.3% trehalose, 3% sucrose, and 10 ppm ABA. For treatment 2, the media for five plates per genotype that had been incubating in the modified development medium BM₃ (containing 5 ppm ABA) were replaced with new modified development medium BM₃ lacking maltose and containing 1% trehalose, 3% sucrose, and 10 ppm ABA. The media in the control plates were replaced again with fresh development medium BM₃. After singulation at 8 weeks followed by the second development media replacement, the embryos were incubated for an additional 4 weeks, resulting in a total development incubation period of 12 weeks.

After development, the embryos were transferred to stratification medium BM₄, and germinated on medium BM₅, as described in EXAMPLE 1. After 6 weeks incubation on germination medium, the embryos were assessed for germination rate, root length, hypocotyls length, and epicotyl length. An embryo was considered a normal germinant if it was in Class 1, which includes the following features: the presence of a 1 mm root (no nubbins), the presence of approximately 5 epicotyl leaves approximately 5 mm long, no large scale hypocotyl ruptures, and the hypocotyl not bent greater than 90 degrees.

Plate yield was determined during singulation at eight weeks by counting the number of embryos present. Plate yields were tracked through to germination to assess germinants/ml medium.

These data were collected for three original treatment sets that followed the same experimental design. Results of two of the three treatment sets (in sum, seven of ten treatments) contain treatments unrelated to the addition of trehalose in late development medium and are not reported here. However, the initial statistical analyses for variation among all of the treatments are reported to provide a more powerful analysis of potential experimental error.

Data for germination rate was analyzed with a generalized linear model with a logit link. Yield, germinants/ml, root length, and hypocotyl length data were analyzed with a mixed linear model. Root length data was first transformed by taking the natural log to stabilize its variance. When variance among all ten treatments was detected, the response means for the trehalose treatments only were compared using Fisher's LSD for multiple comparisons.

TABLE 12
Experimental Conditions
Experimental Design/Media Replacements

| Treatment | 5 week replacement | 8 week singulation/replacement |
|---|---|---|
| 1 | semi-solid BM₃ | semi-solid BM₃ |
| 2 | semi-solid | semi-solid modified BM₃ (-maltose) + |
| | modified BM₃ | 0.3% trehalose, +3% sucrose, |
| | | 10 ppm ABA |
| 3 | semi-solid | semi-solid modified BM₃ (-maltose) + |
| | 1% modified BM₃ | trehalose, +3% sucrose, |
| | | 10 ppm ABA |

Results: As shown in TABLE 13, incubation on development media containing trehalose, sucrose, and low concentration of ABA significantly increased the germination rate of late maturation of Loblolly pine embryos as compared to control treatments that did not contain trehalose. Significant variation was detected among the treatments for effect on germination rate (p<0.0001). The combined means are the least squares means or “ls means” which take into account differences in numbers of embryos between the different genotypes and treatments tested.

As shown below in TABLE 13, LSD multiple comparisons of the combined genotype means for both experimental treatments (providing either 0.3% or 1% trehalose, in addition to 3% sucrose and 10 ppm ABA) indicate a significantly higher germination rate than the control. Although the two experimental treatments did not create significantly different germination rates from each other, the treatment with 1% trehalose induced a higher germination rate than the treatment with 0.3% trehalose. These data indicate a positive correlation between trehalose content during the late maturation stage of embryo development and the ultimate germination rate.

TABLE 13
Germination Rate
Germination Rate (P-value <0.0001)

| | | LSD | | |
|---|---|---|---|---|
| Treatment | LS mean | (α = 0.10) | genotype C mean | genotype D mean |
| 3 | .727 | a | .687 | .745 |
| 2 | .627 | a | .581 | .659 |
| 1 | .439 | b | .377 | .507 |

The data in TABLE 14 indicate that neither treatments of trehalose, sucrose, and reduced ABA significantly effected the yield of the plated loblolly pine embryo genotypes over control treatment. However, as indicated in TABLE 15, when yield data are combined with germination rate to obtain germinants per ml medium, a significant increase in germinants per ml is detected for treatments involving 1% trehalose (treatment 10) over the control treatment and the lower dose of trehalose.

TABLE 14
Yield (P-value = 0.029)

| | | LSD | | |
|---|---|---|---|---|
| Treatment | LS mean | (α = 0.10) | genotype C mean | genotype D mean |
| 3 | 41.20 | a | 42.40 | 39.80 |
| 1 | 40.70 | a | 38.20 | 43.20 |
| 2 | 31.20 | a | 29.40 | 33.00 |

TABLE 15
Germination/ml (P-value = 0.005)

| | | LSD | | |
|---|---|---|---|---|
| Treatment | LS mean | (α = 0.10) | genotype C mean | genotype D mean |
| 3 | 62.89 | a | 62.47 | 63.32 |
| 2 | 39.64 | b | 36.87 | 42.41 |
| 1 | 38.47 | b | 29.19 | 47.75 |

No differences were detected among treatments for effect on root length (p=0.39). However, the data in TABLE 16 indicate that late exposure to trehalose induces greater root length development in genotype C embryos.

TABLE 16
Root Length (P-value = 0.39)

| | | LSD | | |
|---|---|---|---|---|
| Treatment | LS mean | (α = 0.10) | genotype C mean | genotype D mean |
| 3 | 7.86 | NA | 9.72 | 6.56 |
| 2 | 7.23 | NA | 9.40 | 5.67 |
| 1 | 7.09 | NA | 5.21 | 8.79 |

As shown in TABLE 17, treatments of trehalose, sucrose, and reduced ABA significantly increased hypocotyl length over control treatment. Significant variation was detected among the treatments for effect on hypocotyls length (p=0.04). As shown in TABLE 17, LSD multiple comparisons reveal that both experimental treatments resulted in significantly longer hypocotyls than the control treatment. However, the experimental treatments were not significantly different from each other.

TABLE 17
Hypocotyl Length (P-value = 0.04)

| | | LSD | | |
|---|---|---|---|---|
| Treatment | LS mean | (α = 0.10) | genotype C mean | genotype D mean |
| 2 | 11.43 | a | 11.45 | 11.41 |
| 3 | 11.42 | a | 11.68 | 11.17 |
| 1 | 10.61 | b | 10.68 | 10.51 |

In summary, treatments of trehalose, sucrose, and reduced ABA during late maturation of Loblolly pine embryos significantly increase germination rate. Moreover, the data indicate that the positive effects on germination may further increase as the concentration of trehalose in the development media is increased. Additionally, the addition of trehalose to development media had a positive effect on the hypocotyl length of the germinated seeds incubated therein. Finally, the addition of trehalose to development media may have genotype-specific effects on other developmental features such as root length.

### EXAMPLE 5

This Example demonstrates the effects of incubation in late development media containing trehalose, sucrose, and various ABA concentrations on the germination and development of multiple genotypes of Loblolly pine somatic embryos.

Methods:

Somatic embryos of Loblolly pine, genotypes B, C, D, E and F were cultured as described in EXAMPLE 1 up to incubation in the maintenance media. After incubation in maintenance media using the methods described in Example 1, advanced early stage somatic embryos were plated on semi-solid development medium BM₃ (with 25 ppm ABA, 1% glucose and 2.5% maltose) at 25 embryos per plate, 35 plates per genotype. The subsequent experimental design is presented below in TABLE 18. Specifically, five weeks after plating, the medium for 30 treatment plates per genotype was replaced with a modified development medium BM₃ (containing 5 ppm ABA instead of 25 ppm ABA). The medium for the remaining 5 plates per genotype was replaced with fresh development medium BM₃ for control. At 8 weeks after initial plating, the embryos were singulated onto one of six experimental treatment media. All of the experimental treatment media consisted of semi-solid modified development medium BM₃ (lacking maltose and ABA) plus 3% sucrose and 10 ppm ABA. However, as indicated in TABLE 18, the media varied from 0% to 2.5% in their trehalose content. At 8 weeks after initial plating, the control embryos were singulated to fresh development medium BM₃.

Germination rate, yield, germinants per ml medium, root and hypocotyls length were recorded and analyzed as described above in EXAMPLE 4.

Results: The germination results for embryos cultured under the various treatment conditions are provided below in TABLE 18.

TABLE 18
Experimental Conditions and Germination Results (Genotypes C + D)

| | | | Germination |
|---|---|---|---|
| | | | Frequency |
| | | | (combined |
| | | | genotypes |
| Treatment | 5 week replacement | 8 week singulation/replacement | C and D) |
| 1 | Control: semi-solid BM₃ | semi-solid BM₃ | 48.4% |
| 2 | semi-solid modified BM₃ | semi-solid modified BM₃ (-maltose) + | 62.9% |
| | | 0% trehalose, +3% sucrose, 10 ppm | |
| | | ABA | |
| 3 | semi-solid modified BM₃ | semi-solid modified BM₃ (-maltose) + | 55.7% |
| | | 0.3% trehalose, +3% sucrose, 10 ppm | |
| | | ABA | |
| 4 | semi-solid modified BM₃ | semi-solid modified BM₃ (-maltose) + | 60.7% |
| | | 1.0% trehalose, +3% sucrose, 10 ppm | |
| | | ABA | |
| 5 | semi-solid modified BM₃ | semi-solid modified BM₃ (-maltose) + | 63.1% |
| | | 1.5% trehalose, +3% sucrose, 10 ppm | |
| | | ABA | |
| 6 | semi-solid modified BM₃ | semi-solid modified BM₃ (-maltose) + | 63.6% |
| | | 2.0% trehalose, +3% sucrose, 10 ppm | |
| | | ABA | |
| 7 | semi-solid modified BM₃ | semi-solid modified BM₃ (-maltose) + | 75.6% |
| | | 2.5% trehalose, +3% sucrose, 10 ppm | |
| | | ABA | |

As shown above in TABLE 18, a significant increase in germination rate of Loblolly pine embryos is observed as a result of treatments in development medium comprising trehalose, sucrose and lowered ABA levels. As further demonstrated, the positive effect on germination increases with increasing levels of trehalose in the development media.

TABLE 19
Germination Results (Category 1 + 2) Genotypes (B, C, D, E, F)

| | | | Mean | | | |
|---|---|---|---|---|---|---|
| | | | Germination | | | |
| | | | Freq | | | |
| | | | (combined | | | |
| | 5 week | 8 week singulation/ | genotypes | | | |
| Treatment | replacement | replacement | B, C, D, E, F) | α = 0.10 | L90 | U90 |
| 1 | Control: | semi-solid BM₃ | 0.450 | a | 0.395 | 0.505 |
| | semi-solid BM₃ | | | | | |
| 2 | semi-solid | semi-solid modified BM₃ | 0.456 | a | 0.400 | 0.513 |
| | modified BM₃ | (-maltose) + | | | | |
| | | 0% trehalose, +3% | | | | |
| | | sucrose, 10 ppm ABA | | | | |
| 3 | semi-solid | semi-solid modified BM₃ | 0.413 | a | 0.360 | 0.469 |
| | modified BM₃ | (-maltose) + | | | | |
| | | 0.3% trehalose, +3% | | | | |
| | | sucrose, 10 ppm ABA | | | | |
| 4 | semi-solid | semi-solid modified BM₃ | 0.457 | a | 0.400 | 0.514 |
| | modified BM₃ | (-maltose) + | | | | |
| | | 1.0% trehalose, +3% | | | | |
| | | sucrose, 10 ppm ABA | | | | |
| 5 | semi-solid | semi-solid modified BM₃ | 0.461 | a | 0.405 | 0.519 |
| | modified BM₃ | (-maltose) + | | | | |
| | | 1.5% trehalose, +3% | | | | |
| | | sucrose, 10 ppm ABA | | | | |
| 6 | semi-solid | semi-solid modified BM₃ | 0.485 | a | 0.427 | 0.543 |
| | modified BM₃ | (-maltose) + | | | | |
| | | 2.0% trehalose, +3% | | | | |
| | | sucrose, 10 ppm ABA | | | | |
| 7 | semi-solid | semi-solid modified BM₃ | 0.530 | a | 0.469 | 0.589 |
| | modified BM₃ | (-maltose) + | | | | |
| | | 2.5% trehalose, +3% | | | | |
| | | sucrose, 10 ppm ABA | | | | |

L90 and U90 are the lower and upper 90% confidence limits, respectively, for each mean. The column “test at α=0.10” summarizes test results comparing combined means. Means with the same symbol are not statistically different at α=0.10. As shown in TABLE 19, when the 5 genotypes (B,C,D,E,F) are combined, the test for treatment differences was not significant (p=40). However, as shown above in TABLE 18, when genotypes C+D are combined, all of the treatments with trehalose result in improved germination frequency as compared to the control without trehalose. Therefore, some genotype-specific effects are suggested.

While the preferred embodiment of the invention has been illustrated and described, it will be appreciated that various changes can be made therein without departing from the spirit and scope of the invention.

The embodiments of the invention in which an exclusive property or privilege is claimed are defined as follows:

1. A method of increasing germination vigor of conifer somatic embryos produced in vitro, the method comprising:
(a) culturing a plurality of immature conifer somatic embryos for a first incubation period in, or on, a first development medium that comprises less than 0.1% trehalose; and
(b) culturing the plurality of immature conifer somatic embryos treated in accordance with step (a) for a second incubation period in, or on, a second development medium that comprises at least 0.1% trehalose.

2. The method of claim 1, wherein the second development medium comprises between about 0.3% and about 3% trehalose.

3. The method of claim 1, wherein the first development medium comprises at least 10 ppm of abscisic acid.

4. The method of claim 3, further comprising culturing the plurality of immature conifer somatic embryos treated in accordance with step (a) in, or on, an intermediate development medium for an intermediate incubation period before culturing the plurality of immature embryos in the second development medium according to step (b), wherein the intermediate development medium comprises less than 0.1% trehalose and comprises a concentration of abscisic acid less than the first development medium.

5. The method of claim 4, wherein the intermediate development medium comprises less than 5 ppm abscisic acid.

6. The method of claim 4, wherein the intermediate incubation period is between about three weeks and about five weeks.

7. The method of claim 4, wherein the second development medium comprises a concentration of abscisic acid that is not less than the concentration of abscisic acid in the intermediate development medium.

8. The method of claim 7, wherein the second development medium comprises between about 5 ppm and about 25 ppm abscisic acid.

9. The method of claim 1, wherein the second development medium further comprises at least one sugar selected from the group consisting of sucrose, glucose and maltose at a concentration of about 1% to about 4%.

10. The method of claim 9, wherein the second development medium comprises sucrose.

11. The method of claim 10, wherein the second development medium comprises sucrose at a concentration of about 1% to about 4%.

12. The method of claim 1, wherein the first incubation period is a time period sufficient in length for the formation of one or more cotyledonary primordia on a portion of the plurality of immature conifer somatic embryos in the first development medium.

13. The method of claim 4, wherein the combination of the first incubation period and the intermediate incubation period totals a time period sufficient in length for the formation of one or more cotyledonary primordia on a portion of the plurality of immature conifer somatic embryos in the intermediate development medium.

14. The method of claim 1, wherein the second incubation period is a time period sufficient in length for at least a portion of the plurality of immature embryos to reach anatomical maturity.

15. The method of claim 1, wherein the second incubation period is between about three and about five weeks.

16. The method of claim 1, wherein the combination of the first incubation period and the second incubation period totals a time period of at least 12 weeks.

17. The method of claim 1, further comprising singulating a plurality of immature conifer somatic embryos treated in accordance with step (a) and culturing the plurality of singulated conifer somatic embryos on the second development medium in accordance with step (b).

18. The method of claim 17, wherein the singulated immature conifer somatic embryos that are cultured in the second development medium are not in physical contact with one another.

19. The method of claim 1, wherein the conifer somatic embryos are Loblolly pine.

20. A method for producing mature conifer somatic embryos comprising:
(a) culturing a plurality of immature conifer somatic embryos for a first incubation period in, or on, a first development medium that comprises less than 0.1% trehalose for a first incubation period of from 6 weeks to 8 weeks;
(b) singulating a plurality of individual immature conifer somatic embryos cultured according to step (a); and
(c) contacting the plurality of singulated immature conifer somatic embryos for a second incubation period in, or on, a second development medium that comprises at least 0.1% trehalose for a second incubation period sufficient in length for at least a portion of the singulated immature conifer somatic embryos to reach anatomical maturity.
